(19) United States
(12) Patent Application Publication
(10) Pub. No.: US 2013/0012812 A1
(43) Pub. Date: Jan. 10, 2013

(54) FUNCTIONAL AND PHYSICAL IMAGING BY SPECTROSCOPIC DETECTION OF PHOTO ABSORPTION OF PHOTONS AND SCATTERED PHOTONS FROM RADIOACTIVE SOURCES OR DIFFRACTED X-RAY SYSTEMS

(75) Inventor: Edward J. SEPPI, Portola Valley, CA (US)

(73) Assignee: VARIAN MEDICAL SYSTEMS, INC., Palo Alto, CA (US)

(21) Appl. No.: 13/177,402

(22) Filed: Jul. 6, 2011

(51) Int. Cl. A61B 6/00; G01N 23/223; G01N 23/201

(52) U.S. Cl. 600/431; 378/44

(57) ABSTRACT

An apparatus to examine a target volume in a patient includes an x-ray source generating a first x-ray beam targeting the target volume, and a detector which is placed at an angle less than 180 degrees relative to a beam path of the first x-ray beam to receive a second x-ray beam generated from the first x-ray beam interacting with the target volume. A method to image a target volume in a patient includes directing a first x-ray beam generated from an x-ray source at the target volume, wherein a second x-ray beam is generated by an interaction of the first x-ray beam with the target volume, detecting the second x-ray beam using a detector that is placed at less than 180 degrees relative to a path of the first x-ray beam, and obtaining spatial and temporal information of the target volume using the detected second x-ray beam.

### FIELD

This application relates generally to apparatus, system, and methods for medical imaging and, more specifically, to a new technique for x-ray medical imaging and functional evaluation.

### BACKGROUND

There are many medical imaging techniques currently used for diagnosis, include computerized tomography (CT), nuclear spectroscopy, magnetic resonance imaging (MRI), positron emission tomography (PET), which can provide the non-invasive medical image of a target in the patient. Some of the above techniques require radioactive imaging agents to be used in conjunction, such as PET. The radioactive imaging agents interact with the tissue(s) in the target region of the patient, thereby allows the tissue(s) that contains the agent to be detected using a detector.

The x-ray based imaging techniques, such as CT, detects the x-rays penetrated, attenuated, and/or scattered by the target region, on a medium that is opposite of the x-ray source, such as x-ray sensitive film or photonic detector. These x-ray based imaging techniques, however, do not account for all of the photons directed at the target region. Applicant of the subject application determines that secondary x-ray beams, such as scattered x-ray, may hold valuable imaging data that has not been utilized.

### SUMMARY

In accordance with some embodiments, an apparatus to examine a target volume in a patient includes an x-ray source generating a first x-ray beam targeting the target volume, and a detector which is placed at an angle less than 180 degrees relative to a beam path of the first x-ray beam to receive a second x-ray beam generated from the first x-ray beam interacting with the target volume.

In accordance with other embodiments, a method to image a target volume in a patient includes directing a first x-ray beam generated from an x-ray source at the target volume, wherein a second x-ray beam is generated by an interaction of the first x-ray beam with the target volume, detecting the second x-ray beam using a detector that is placed at less than 180 degrees relative to a path of the first x-ray beam, and obtaining spatial and temporal information of the target volume using the detected second x-ray beam.

Other and further aspects and features will be evident from reading the following detailed description of the embodiments.

### BRIEF DESCRIPTION OF THE DRAWINGS

The drawings illustrate the design and utility of various embodiments of the present application, in which similar elements are referred to by common reference numerals. In order to better appreciate how advantages and objects of the present application are obtained, a more particular description will be rendered by reference to specific embodiments thereof, which are illustrated in the accompanying drawings. Understanding that these drawings depict only typical embodiments and are not therefore to be considered limiting of its scope. The present application will be described and explained with additional specificity and detail through the use of the accompanying drawings in which:

FIG. 1 is a schematic illustrating an apparatus of scatter x-ray detection in accordance with some embodiments;

FIG. 2A is a schematic illustration of imaging a volume of breast tissue in accordance with some embodiments;

FIG. 2B is a schematic illustration of imaging a volume of abdominal tissue in accordance with some embodiments;

FIG. 3 is an example of an energy spectrum diagram generated using an ²⁴¹Am source with ¹²³I imaging agent in accordance with some embodiments;

FIG. 4 is a schematic illustration of an example of a pencil beam source as the radiation source and pencil secondary beam detection mode in accordance with some embodiments;

FIG. 5 is an example of an incident beam detected spectra of photo-absorption and emission using an ²⁴¹Am source in accordance with some embodiments;

FIG. 6 is a schematic illustration of an example of a pencil beam source as the radiation source and planar secondary beam detection mode in accordance with some embodiments;

FIG. 6A is an alternative schematic illustration of a pencil beam source as the radiation source and planar secondary beam detection mode, and the planar collimator, in accordance with some embodiments;

FIG. 7 is a schematic illustration of CT imaging using a planar beam source as the radiation source and planar secondary beam detection mode in accordance with some embodiments;

FIG. 8 is a schematic illustration of an example of a planar beam source as the radiation source and planar secondary beam detection mode in accordance with some embodiments; and

FIG. 9 is a schematic illustration of an apparatus configuration in accordance with some embodiments.

### DETAILED DESCRIPTION

Various embodiments of the present application are described hereinafter with reference to the figures. It should be noted that the figures are not drawn to scale and elements of similar structures or functions are represented by like reference numerals throughout the figures. It should also be noted that the figures are only intended to facilitate the description of specific embodiments. They are not intended as an exhaustive description of the present application or as a limitation on the scope of the present application. In addition, an aspect or a feature described in conjunction with a particular embodiment is not necessarily limited to that embodiment and can be practiced in any other embodiments of the present application.

This application provides for an apparatus and a method to measuring the spatial distribution, temporal attributes, and/or functional attributes of a material of interest. Unlike apparatus and methods that measure photonic information received directly opposite of the x-ray source, which measures the x-ray photons travelling through the material of interest, embodiments of the apparatus and method described herein measure the x-ray photons that are scattered or generated by the incident x-ray photons in the material of interest (such as tissue). Embodiments of the system and method described herein uses incidental excitation radiation from a source external to the material of interest, which may include the target tissue and/or an agent such as injected contrast agent and/or implanted objects, to produce secondary radiation having different characteristics and different beam paths from the incident radiation. This secondary radiation, having different attributes from that of the incident radiation, is produced by interaction of the incident radiation with the tissue and/or an agent such (as contrast agent, implanted material, or combination thereof). The secondary radiation is detected external to the material of interest, and may be analyzed to determine its source location, directionality, type, spatial distribution, or other attributes. Information about the incident radiation and detected secondary radiation (both of which may be of more that one type) may be used to determine positional, geometric, functional attributes, and temporal attributes of the material of interest. The spatial distribution may include the existence, density, location, function, and shape of the material of interest in the target volume being studied. The temporal attributes (which may be obtained by continuous monitoring or monitoring the same point of interest over different points in time) may include the rate of build-up and clearance, the pattern of flow, changes in existence of, density, location, function, shape and location of material nodules, and other attributes of the various types of molecules present in the material of interest. An example of detecting such temporal attributes may be accomplished by observing the change of the various types of molecules present over time (e.g., measuring the change of the amount of glucose in the tissue over time). The functional attributes may be determined by simultaneous observation of two or more attributes, such as by observing two or more types of secondary radiation emitted from the material of interest, projecting two or more types of excitation radiation onto the material of interest, injecting two or more types of contrast agent or tissue, or any combination thereof. For example, in some embodiments, functional attributes may be determined by observing the change in ratio or the function of two different types of tissue or structure (e.g., tissue, fat, bone, lung, angiogenesis liquid, solid) at a point of interest over time represents one or more human body function(s)) in the material of interest. The detected spatial distribution, temporal attributes and functional attributes can be arbitrarily combined in some embodiments.

Embodiments described herein involve using an incidental excitation x-ray radiation that interacts with material of interest, which may include tissue and/or an agent (e.g., iodine contrast agent, gadolinium, gold, bismuth) in a target volume. In other embodiments, instead of using x-ray radiation, other types of beams, such as a proton beam, may be used. Thus, as used in this specification, the term “radiation” is not limited to x-ray radiation, and may refer to other types of beam that radiate, such as a proton beam. High Z elements or elements with high electron density that are naturally occurring in tissues may also act as an agent in some embodiments. The spatial, temporal, and functional attributes can be determined using: a) photo absorption of incidental excitation x-ray beam and subsequent emission of characteristic photons (e.g., K-alpha and K-beta radiation) by the material of interest, as well as b) secondary radiation generated from Compton scatter of incidental excitation x-ray beam interacting with tissue.

Embodiments of the apparatus and method described herein involve using an excitation beam that interacts with material of interest to generate a secondary radiation. The material of interest includes an atom or a particle in target tissue with which the incident radiation interacts. In some embodiments, the material of interest may be a particle occurred naturally in the tissue. In such cases, the excitation beam interacts with the atoms and electrons in the tissue itself. These interactions yield secondary radiation which can be measured to determine various information. The information can be used either alone, or with information obtained using an agent, such as a contrast.

In other embodiments, the material of interest may be an agent (or more than one agent) externally introduced into the tissue for enhancement of detection. The agent may be particle(s) or fluid. By means of non-limiting examples, the agent may be administered to the patient using various methods, such as by injection, absorption, implant, or attachment (e.g., a material attached to a molecule, which through body function, attaches itself to cancer, hypoxic tissue, a particular tissue etc.). The agent may be any material other than the tissue. An exemplary agent would be a contrast agent that has properties suitable for detection such that if the external radiation is an x-ray radiation at an appropriate energy level, the external radiation will interact with the agent to produce photo absorption-emission radiation and Compton scatter radiation.

In further embodiments, the material of interest may be both tissue and an agent.

In still further embodiments, the material of interest may be an atom of a particle in a non-biological volume to be examined.

As discussed, the incident radiation interacts with material of interest in a manner where the measured spatial, functional and/or temporal data may provide important information. The excitation beam (or incidental beam) is a radiation beam with sufficient intensity (number of photons) and specific radiation quantum attributes (e.g., quantum energy (such as photon energy), quantum spectrum (such as number of photons in a small band of energy found in the beam), and other properties such as polarization of the radiation), which may be quasi-monoenergized, collimated, directed, and/or controlled. The source of the excitation beam is external to the material of interest and is directed to pass into the material of interest. For example, the radiation is optionally quasi mono-energetic and at a suitable quantum energy. Such quasi mono-energetic radiation can be created by x-ray treated with crystal diffraction that may be shaped into a pencil beam, a fan beam, flood beam, a cone beam, and other arbitrarily shaped beams. In other embodiments, proton beam, neutron beam, or other particle beams may be used as the excitation beam. In some embodiments, the excitation beam may be generated using crystal (in the radiation source) to create a refracted beam at an angle. By adjusting the angle, the excitation beam with a certain desired energy level may be generated. The orientation of the crystal in the radiation source may be adjusted in some embodiments.

The secondary radiation is radiation generated as a result of interaction of the excitation radiation with the material of interest. This secondary radiation is externally detected and analyzed to determine the source location, directionality, temporal, and/or type attributes of the material of interest. In some embodiments, the secondary radiation is detected with apparatus which may be collimated to sense its directionality and different aspects of the radiations attribute (e.g., intensity (such as number of photons), energy (such as photon energy), spectrum (such as number of photons in a small band of energy found in the beam), and other attributes, such as the “spin” or polarization of the radiation). As an example, the secondary beam may be collimated which defines the attributes of cross sectional size and shape, and the direction for the scattered x-ray photons to be analyzed. In some embodiments, the beam's energy signature may be determined by x-ray spectrum analysis. Information about beam energy spectrum and/or the energy of particular photons may be used to evaluate attributes of the material of interest. In some embodiments, if proton beam is used as the excitation beam, the secondary radiation of interest maybe generated by nuclear decays.

Embodiments of the system described herein include a photon detection system. In some embodiments, the detection system may include an array of spectrum sensitive x-ray detectors for analysis of the scattered photons. In other embodiments, the detection system may also have collimation directivity control and/or photon energy spectrum analysis capability, but the system is not limited to the exemplary capabilities it may have. Also, in any of the embodiments described herein, the system may further include a calibration and correction apparatus for performing calibration and correction procedures to attain results of good accuracy. Furthermore, in any of the embodiments described herein, the system also includes a processor (which may be implemented using a computer) for analyzing the acquired signals.

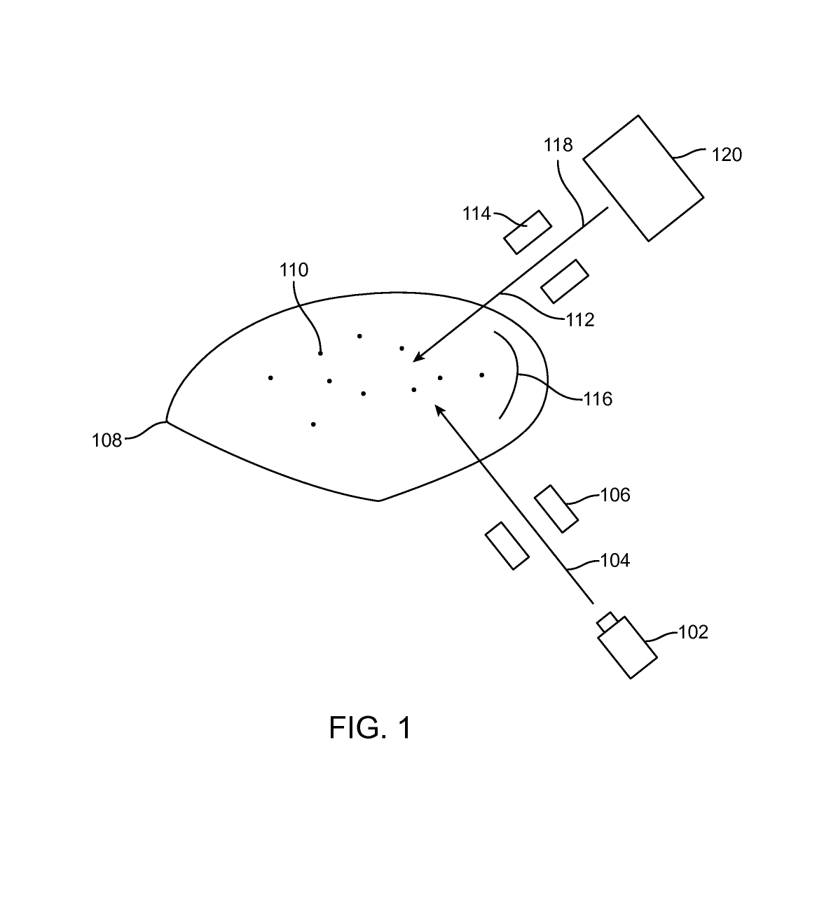

FIG. 1 illustrates a system 10 in accordance with some embodiments. The system 10 includes a radiation source 102, an incidental collimator 106, an exit beam collimator 114, and a spectrum sensitive photon detection system 120. The radiation source 102 is configured to provide an excitation beam 104. The excitation beam 104 from a radiation source 102 is directed toward a material of interest 110 within a target volume 108. Optionally, there can be two or more target volumes in the path of the excitation beam. There can also be more than one materials of interest within one or more target volume in the path of the excitation beam. The excitation beam 104 is optionally collimated by incidental collimator 106 and the secondary radiation 112 (e.g., resulted from the photoelectric and Compton interactions) is detected and analyzed. Secondary radiation 112, which travels in directions different from that of the incident excitation beam (e.g., at an angle 116) and have a different quantum energy, is produced from the excitation beam 104 interacting with the material of interest 110. For the secondary radiation produced by photoelectric effect, the quantum energy of the secondary x-ray is highly dependent on the property of the atom (e.g., the atomic number) that the incident excitation beam impinges upon. For the secondary radiation produced by Compton effect, the interaction is primarily with the electrons in the material. The secondary quantum energy is dependent on the scatter angle. The excitation beam may impinge upon the atom of the normal tissue, and/or the imaging agent, or their respective constituent electrons. For the secondary radiation produced by Compton scatter effect, the quantum energy of the secondary x-ray is dependent on electron constituents of the material of interest and the angle at which the secondary x-ray is detected. As an example, the secondary x-rays are emitted and detected at an angle 116, which may be approximately 30 to 170 degrees relative to the path of excitation beam 104. The secondary x-rays 112 may be collimated by exit beam collimator 114, and the collimated analysis beam 118 is detected and analyzed. The combination of collimators 106 and 114 defines the target volume from which the secondary radiation is emitting. The analysis beam 118 may be measured by the spectrum sensitive photon detection system 120. In some embodiments, the analysis beam 118 may include photons which are primarily produced by the excitation x-ray beam interacting with the material of interest in the target volume defined by collimators 102 and 114. In other embodiments, the system 10 may not include either or both of the collimators 106, 114.

An embodiment to examine a target volume of tissue using the system 10 will now be described. The location and shape of the target volume may be defined by the effective crossing point and cross sectional area of the pencil incident beam and the detected envelope. For example, with respect to FIG. 1, the incident collimation 106 defines a surface which contains the incident radiation. The exit beam collimation 114 defines another surface through which the exit radiation must pass. These two surfaces may intersect to form an enclosed volume. This may define a target volume in some embodiments, which has a location and a shape. In some embodiments, one or more incident pencil beams, one or more secondary detection envelopes, and one or more target volumes may be used. In some cases, the detection envelope may have a detection area that is larger than the cross section of the geometric detection pencil. The detection envelope may include detection envelopes such as secondary radiation spectrum (quantum intensity in envelopes of energy), and secondary quantum polarization. Various sources and detector envelopes are possible in different embodiments. A source envelope maybe an incident beam having quasi mono-energetic x-rays with some central energy, or two or more different central energies. It may be polarized or un-polarized, coherent or incoherent, pulsed or continuous. Also, in some embodiments, the sensitivity envelope of the detector maybe energy sensitivity, polarization sensitivity, etc.

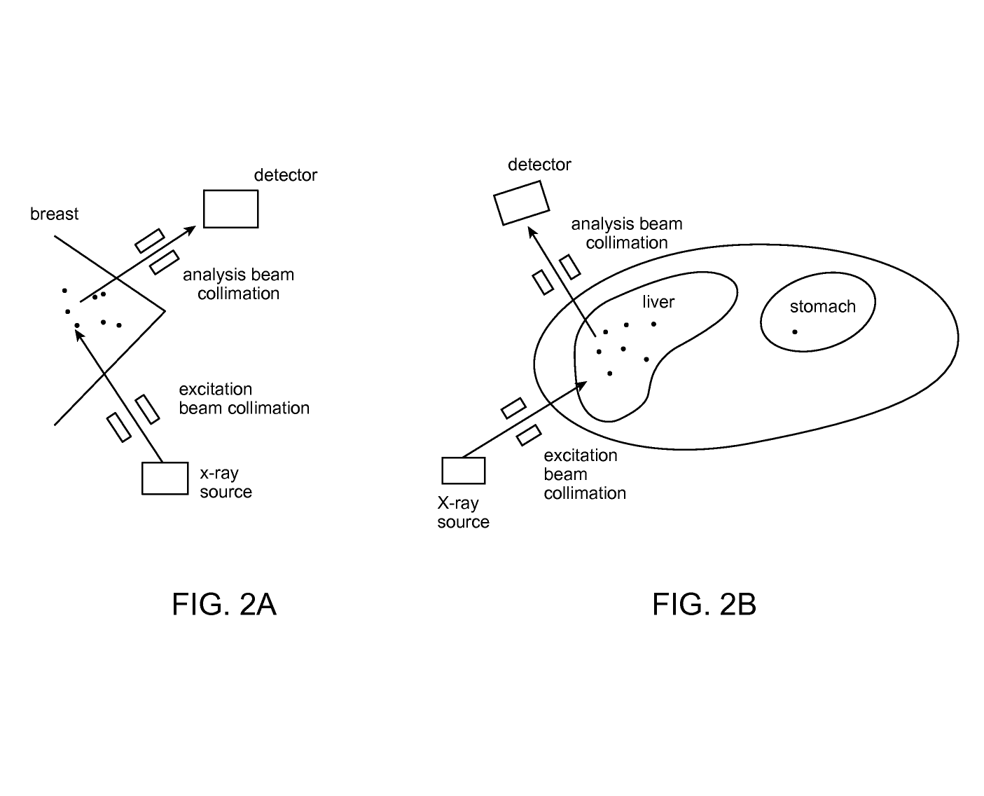

An imaging agent may be optionally administered in the target volume. If an imaging agent is not administered, the measurement depends on the different materials naturally occur in the target volume. The volume may be the breast (or breast with chest wall), liver, prostate, kidney, lung, or other anatomical site of interest (an embodiment to examine breast is shown in FIG. 2A and another embodiment to examine abdomen is shown in FIG. 2B). In general, an excitation beam is arranged to target a voxel in the site of interest, and secondary beams originate from the voxel are generated after the photons from the excitation beam interact with the voxel. The interactions that generate secondary beams are: 1) the photo absorption and photo emission (also known as photo absorption and emission radiation, or photo secondary radiation) by one of the constituents of the voxel (e.g., the constituent(s) may be an agent, or otherwise a naturally occurring material); and 2) the Compton scattering of the x-ray photons by the electron constituents of the voxel.

During use of the system 10, the excitation beam 104 is directed to the target region, and secondary beam 112 (which in various embodiments may or may not be collimated), is then detected by the detector 120. The detector 120, which may be a single-count, quantum energy sensitive detector, is configured to provide signals to a processor (coupled to the detector 120) in response to the detected secondary beam 112. The processor is configured to create an energy spectrum using the signals from the detector 120. In some embodiments, the signals from the detector 120, and information (such as the spectrum) generated by the processor may be stored in a non-transitory medium for later processing, and/or may be displayed in a screen for allowing a user to examine the data.

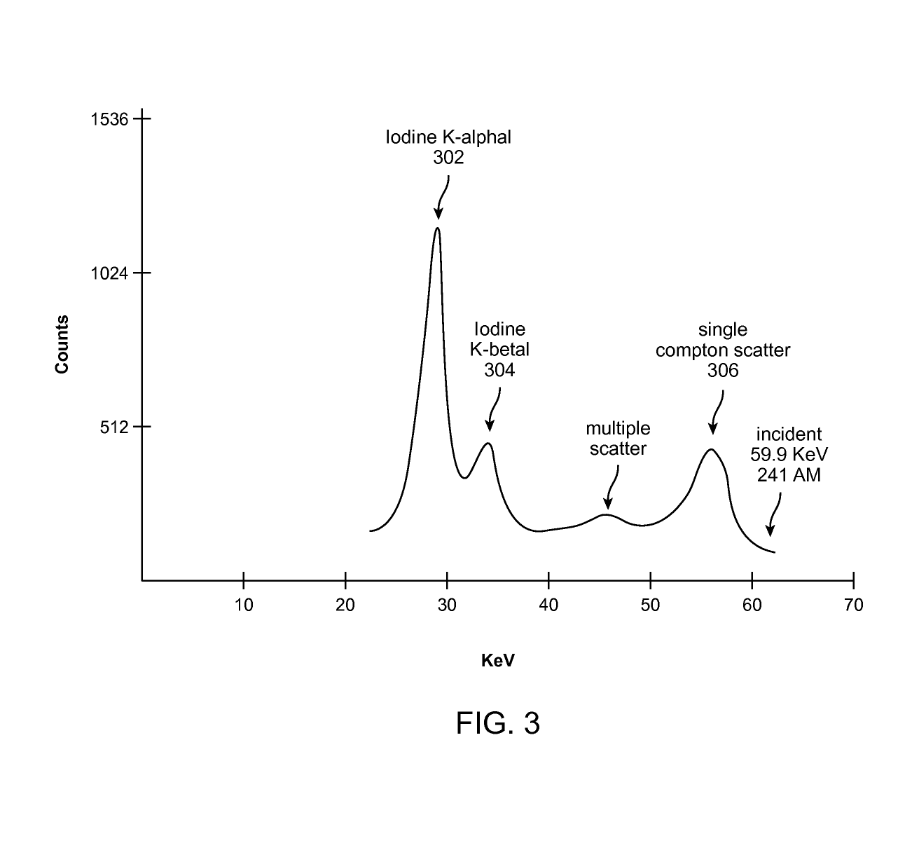

The energy spectrum of a voxel administered with ¹²³I showing both interactions (photo absorption and emission, and Compton scattering) is shown in FIG. 3. As discussed, photo absorption and photo emission by the imaging agent contribute to the creation of the secondary beams 112. As the photons from the excitation beam 104 are absorbed by the atoms of the imaging agent, the electrons of the atoms are elevated to an excited state (photo absorption) followed by rapid decay and emission of radiation photons (photo emission). The emitted radiation photons exhibit the energy characteristics of the excited atom. For example, if the administered agent is iodine and the energy of the excitation beam is at ˜59.9 keV from an ²⁴¹Am source in the radiation source 102, the energies of the fluorescence radiation photons K-alpha 1 (shown as peak 302 in FIG. 3) and K-beta 1 (shown as peak 304 in FIG. 3) from photo interaction of the incident radiation with the Iodine are ˜28.7 keV and 32.4 keV, respectively. The excitation beam 104 also interacts with the atoms of the tissue, but since the atomic number of the atoms of the tissue is lower, the emitted photons have the energy of <10 keV. The emitted radiation photons are emitted in all directions.

Thus, as illustrated above, based on the values of the energy of the fluorescence radiation photons in the graph, the material in the target region may be identified (because different materials may produce different unique energy levels for the photonic energy Ep).

Also, as discussed, compton scatter of the x-ray photons by the electron constituents of the voxel is another phenomenon that contributes to the creation of the secondary radiation in the secondary beam 112. The phenomenon of Compton Scatter is well known in the art. In general, the x-ray photons interact with the constituents (e.g., electrons) in the voxel (in presence or absence of the agent), and are scattered in new directions and lose energy in the process. The number of electrons in the voxel is not significantly changed by the presences or absence of the agent. The energy of the scattered photons is dependent on the scatter angle, which can be calculated by the following equation:

Energy of Scatter Photon Ec=0.51 MeV/((1−cos(scatter angle))+(0.51 MeV/Energy of Incident Photon))

In the above equation, the scatter angle is the angle between the path of incident beam 104 and the path of scatter beam 112. All of the Compton scattered radiation in a cone with its axis in the direction of the incident pencil beam have the same energy quantum energy since the scatter angle is the half angle of the cone. For example, if the incident photons have the energy of ˜60 keV, using the information above, the energies Ec of Compton scattered photons (shown as peak 306 in FIG. 3) for scatter angles 75 degrees, 90 degrees, and 105 degrees are 55.1 keV, 53.6 keV, and 52.2 keV respectively.

Also, in some embodiments, a processor (coupled to the detector 120 for receiving data from the detector 120) may be configured to determine the ratio Ep/Ec from the graph. If Iodine is used as the agent, then the ratio Ep/Ec correlates with the ratio of Iodine mass/tissue mass at the target region. In some cases, the ratio Ep/Ec may be calculated at every prescribed time interval (e.g., every 1-2 seconds), and the resulting ratios may be stored in a non-transitory medium for later processing. For example, the ratios may be presented in a plot of time (in the x-axis) versus ratio (in the y-axis), which may be displayed in a screen. The plot may be used to examine leakage of the agent at the target region (e.g., how fast the agent at the target region is leaking).

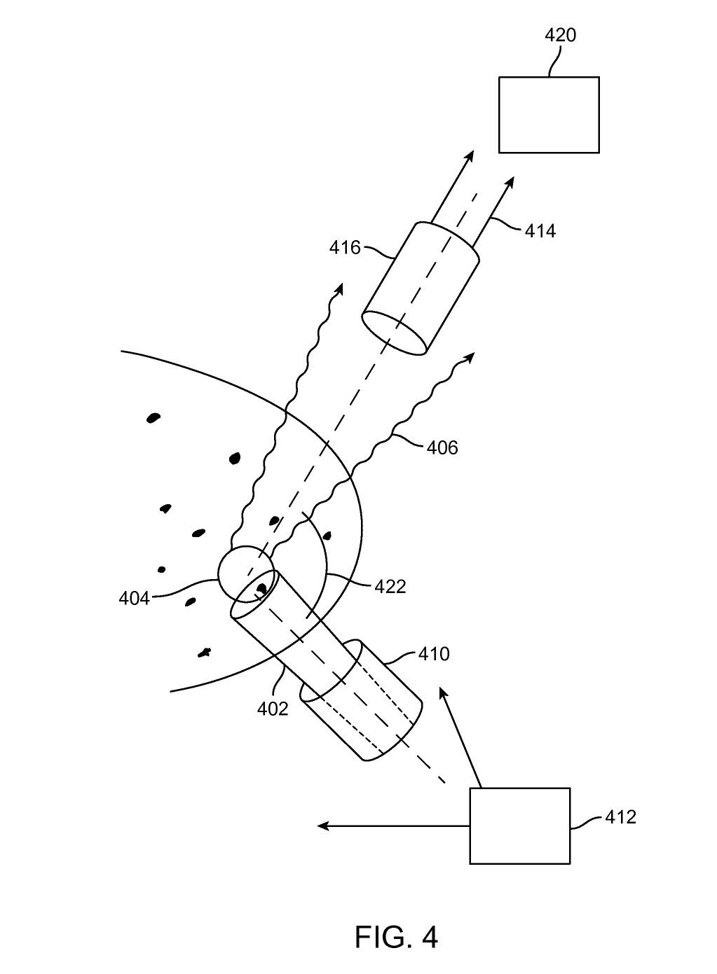

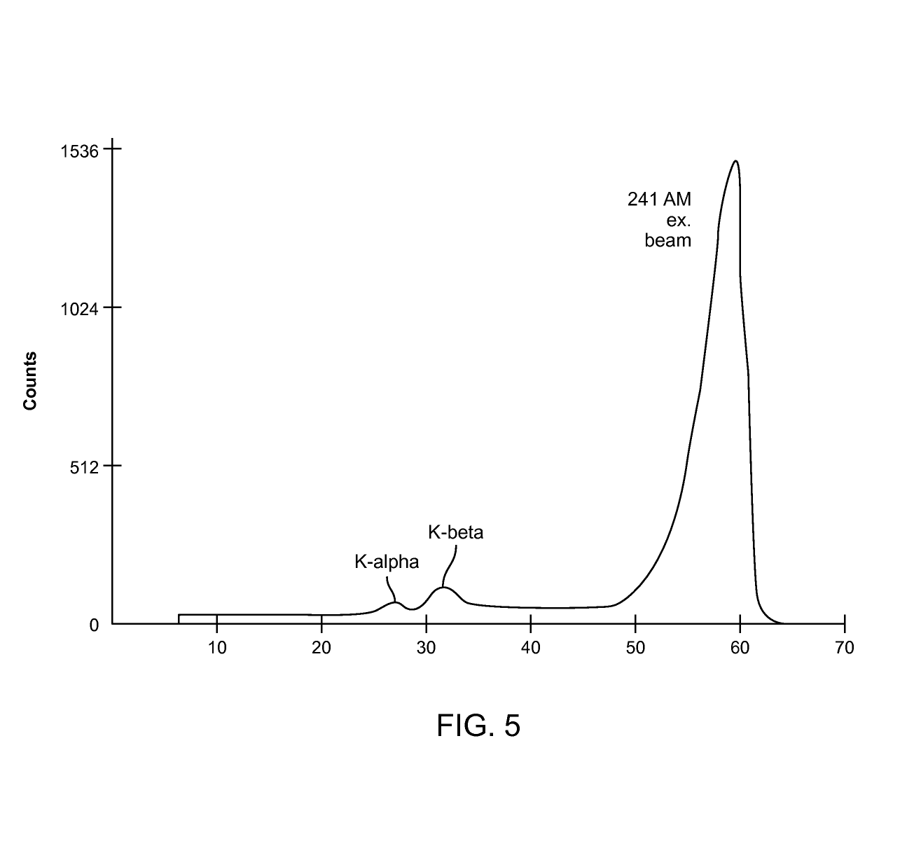

In any of the embodiments described herein, the excitation beam 104 may be a pencil beam, and the secondary beam may also be a pencil beam (excitation pencil beam-secondary pencil beam detection mode). Also, in some embodiments, other shaped incident and exit beams may be achieved as groups of multiple pencil beams leading to any desired shape for the incident collimation and/or exit collimation. FIG. 4 illustrates a detection system that utilizes excitation pencil beam and detected secondary pencil beam in accordance with some embodiments. In this embodiment, the excitation beam is emitted from x-ray source 412 (which may be an example of the source 102 of FIG. 1) and collimated to circular or rectangular shape by a first collimator 410 (which may be an example of the collimator 106 of FIG. 1) having nominal diameter (e.g., ˜3 mm), which may be referred to as pencil beam. The excitation pencil beam 402 may have monochromatic or multi-chromatic spectral characteristics. The source 412 includes an electronic x-ray generated by an x-ray tube and crystal diffraction apparatus, which may be a quasi energetic source generated by an electronic device or it may be radioactive substance. This configuration allows the quantum energy and the intensity of the pencil beam 402 to be arbitrarily selected, and the spectral characteristics to be optimized. As an example, FIG. 5 shows the spectral distribution of an excitation beam using ²⁴¹Am as the radiation source 412, measured with an energy sensitive Hgl detector system (or may also be a CdTe detector or a detector having any of other energy sensitive photon sensing materials), which can detect x-ray photons with multiple energy levels. In other embodiments, the radiation source can be other materials.

Returning to FIG. 4, the secondary beams 406 are generated by interaction of the excitation beam 402 with a voxel 404 (e.g., a volume of tissue and/or imaging agent optionally administered within the target volume). The voxel 404 and its location is defined by the intersection of the excitation beam 402 and the secondary beam 406 within the target volume. In this example the secondary beams 406 are generated in a way that is similar to a light bulb radiating light in all directions. The voxel 406 may be “viewed” by collimation of the secondary beam 406 at by a second collimator 416 (which may be an example of the collimator 114 of FIG. 1) down to size of pencil beam (secondary analysis beam 414) and analyzed by the detector 420 (which may be an example of the detector 120 of FIG. 1). Essentially, only a sample of the secondary radiation is analyzed.

The source incident beam 402 and the detected secondary beam 414 may be adjusted (e.g., scaled, positioned, shaped, etc.) to view voxels of various shapes, sizes, and locations through adjustment of collimation (410 and/or 416), source 412, and/or the size of the detector 420. The detector 420 may detect photons with different quantum energy levels and is placed at an angle 422 relative to the excitation beam source 412 (e.g., the angle between the path of the excitation beam 402 and the path of the secondary radiation 414).

The secondary analysis beam 414 has primarily two components. One of which is the scattered beam generated from photo absorption of the excitation pencil beam 402 by the imaging agent. The photo scattered beam has intensity that is proportional to the amount of imaging agent in the voxel, density of materials of the voxel, and intensity of the excitation beam 402. The photo scattered beam's quantum energy spectrum is dependent on the atomic number of materials in the voxel. Another component of the secondary radiation 414 is the Compton scatter radiation, which may be detected by the multi-energy detector in addition to the scattered beam. The Compton scatter is primarily dependent of the electron density in the voxel. Its quantum energy spectrum depends on the angle of scatter and incident beam quantum energy spectrum. In some embodiments, spatial and temporal information may be derived from detected secondary beams 414, which are analyzed to produce medical image for diagnostic and/or treatment. To acquire sufficient data for volumetric spatial and temporal information, the excitation beam 402 may raster scan the entire target volume voxel by voxel (the detector 420 and secondary beam collimator 416 may move dependently from the excitation beam source 412). In some embodiments, one or both of the incident source beam and the exit beam detection may be rastered. In other embodiments, individual point(s) of interest may be selected, in which case, the scanning may not be required.

Relative temporal information may be obtained by measuring two or more points of interest simultaneously, or measuring a point of interest at two or more time points. As an example, using two or more detectors and/or two or more incidental beams, measuring two or more points of interest simultaneously may yield information of the tissue, agent spatial distribution, and/or electron density (or function) at that time point. As another example, measuring spatially distribution of a point of interest at two or more time points may yield the temporal information of how the spatial distribution (and/or electron density) of an agent changes over time. In some embodiments in which more than one excitation beam and more than one corresponding detector are used, the amount of raster scan may be reduced.

In any of the embodiments described herein, a processor may be configured to perform a process (e.g., a reconstruction process) to analyze the acquired data and transform the data to a result in a form appropriate for use in medical or non medical (e.g. industrial, security, etc.) application. Any of the acquired data, information, and results described herein may be stored in a non-transitory for later processing/use, and/or for display on a screen.

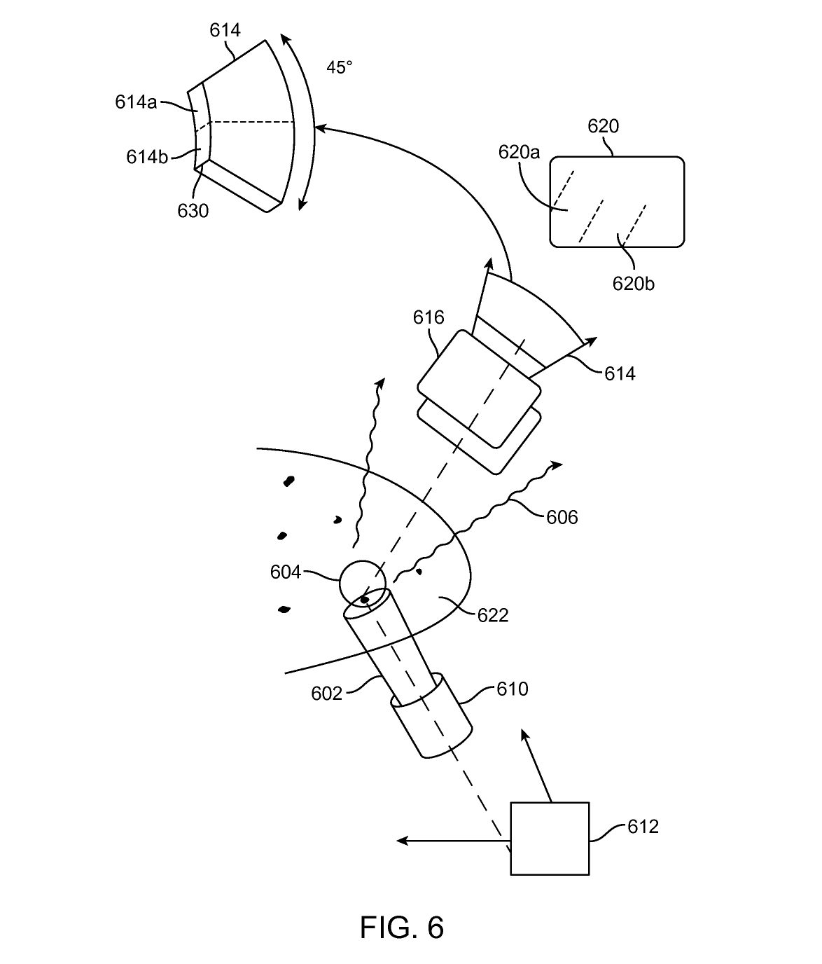

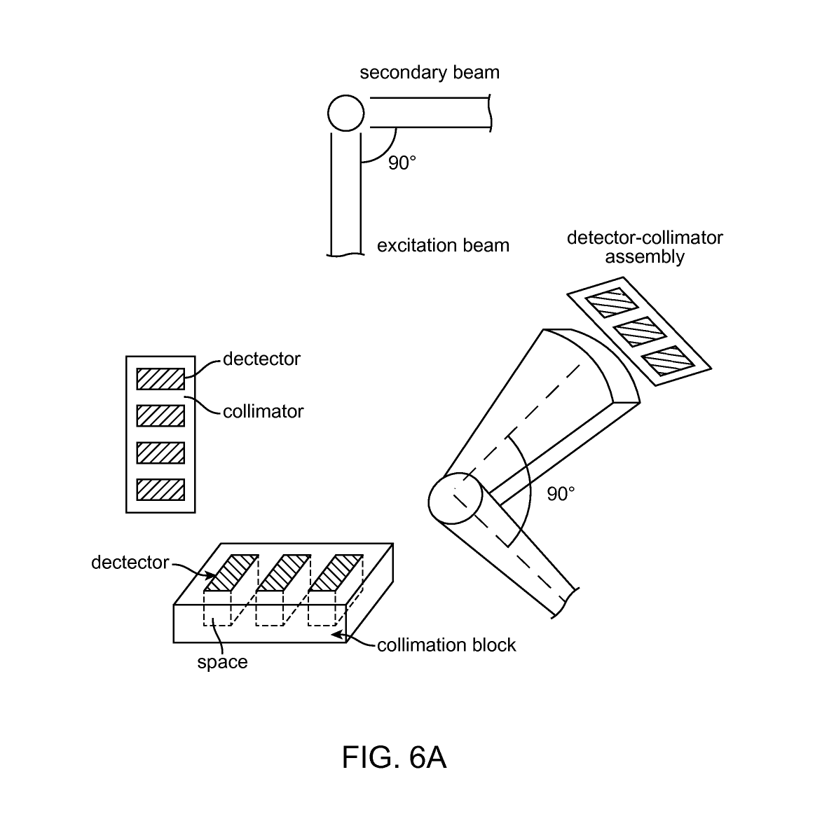

In any of the embodiments described herein, the excitation beam 104 may be a pencil beam, and the secondary beam may be a planar beam (excitation pencil beam-secondary planar beam detection mode). FIG. 6 illustrates a detection system that utilizes excitation pencil beam and detected secondary planar beam in accordance with some embodiments. In this embodiment, the excitation beam is emitted from x-ray source 612 (which may be an example of the source 102 of FIG. 1) and collimated by a first collimator 610 (which may be an example of the collimator 106 of FIG. 1) into excitation pencil beam 602. The excitation pencil beam 602 may have monochromatic or multi-chromatic spectral characteristics. The excitation pencil beams' energy and intensity may also be optimized through Bragg diffraction, crystal selection, and/or collimation. The secondary beams 606 are generated by interaction of the excitation beam 602 with a voxel 604 of tissue and imaging agent within the target volume. The secondary beams 606 are collimated by a planar collimator 616 (which may be an example of the collimator 114 of FIG. 1), and are received by the detector 620 (which may be an example of the detector 120 of FIG. 1). In the illustrated embodiments, the shape of the secondary planar analysis beam 614 resembles a sector (e.g., a plane figure bound by two radii and the included arc of a circle) having a thickness 630. The thickness of the sector-shaped secondary planar beam 614 is approximately the diameter of the excitation pencil beam 602 and the arc of the sector is 45 degrees. In some embodiments, collimator 616 may be sectioned into multiple ports to produce respective beams 614a, 614b, with detector 620 having respective detector regions 620 a, 620b for sensing the radiation beams 614a, 614b after passing through a subject. Although two beams 614a, 614b are shown, in other embodiments, the number of beams may be more than two. In other embodiments, the shape of the planar beam 614 may have different shapes, and/or different thicknesses, from those described. Also, in other embodiments, the detector 620 may have different configurations.

In the illustrated embodiments, the shape of the voxel is approximately spherical (e.g., radius defined by the diameter of the excitation pencil beam) and defined by a volume which is the intersection of the plane of the excitation pencil beam cross-section and the collimation plane cross-section or ports thereof. This mode may detect the spatial and temporal data of the voxel at a higher sensitivity than the excitation pencil beam-secondary pencil beam detection mode. In some embodiments, with multi collimator ports, multiple voxels may be investigated simultaneously. Also, in some embodiments, a multiport collimator, in combination with a stacked multiple sectors detector, may be used to view individual voxels along the entire length (or a portion) of the incident pencil beam 602, thereby performing a line scan with high sensitivity. To collect volumetric data, raster scanning may be performed. The raster scan can either be a point by point scan (three-dimensional scans of a volume), or line by line scan (two-dimensional line scans of a volume). Use of multiple excitation pencil beams (e.g., multiple radiation sources) and multiple detectors can reduce the raster scan performed.

The embodiments of FIG. 6 may have different variations. For example, in a configuration where the direction of the excitation pencil beam 602 is perpendicular to the plane of the secondary planar beam 614 (e.g., detector is placed at 90 degree angle relative to the x-ray source 612, shown in FIG. 6A), the data collected by the detector 620 detecting the planar beam 614 is related to the line integral of the interactions along the excitation pencil beam path. In other embodiments, instead of 90°, the detector 620 may be placed at any angle relative to the x-ray source 612. In some cases, the line integral may be used to survey the boundaries of a sub-set volume having high density of imaging agent within the target volume. Also, during an imaging procedure, after the boundaries are surveyed, the imaging system may be reconfigured to operate in a different mode to investigate detail agent distribution in a more restricted volume. For example, in some embodiments, the collimator/detector may be moved closer or further from the target object, thereby changing the size/shape of the observed voxel. In other embodiments, in addition or in the alternative to moving the collimator/detector, the collimator components may be mechanically adjusted to make the ports smaller or larger, e.g. the height 630 may be changed. These are examples of changes in the detection collimation and detector properties. Similar changes may be made in the source collimation and source properties in other embodiments.

In any of the embodiments described herein, a processor may be configured to perform a process (e.g., a reconstruction process) to analyze the acquired data and transform the data to a result in a form appropriate for use in medical or non medical (e.g. industrial, security, etc.) application. Any of the acquired data, information, and results described herein may be stored in a non-transitory for later processing/use, and/or for display on a screen.

In any of the embodiments described herein, the excitation beam 104 may be a planar beam, and the secondary beam may be a pencil beam (excitation planar beam-secondary pencil beam detection mode). In this embodiment, the excitation beam 104 is collimated into excitation planar beam 104. The excitation planar beam 104 may have monochromatic or multi-chromatic spectral characteristics. The excitation planar beams' energy and intensity may also be optimized through Bragg diffraction, crystal selection, and collimation. The secondary beams 112 are generated by interaction of the excitation planar beam 104 with a voxel of tissue and imaging agent within the target volume. The secondary beams 112 are collimated by a pencil collimator. This detection mode may be used to view individual voxels along the entire length, or a portion of the length, of the secondary beam 112. For an example, the detector 120 may be placed stationary relative to the target volume and the x-ray source 102. The x-ray source 102 may be translated in any direction which moves the intersection point along the axis of beam 112 to collect data of voxels within the target volume that are along the path of the detection beam. For example, in some embodiments, the source 102 may translate in either or both directions that are parallel to the axis of beam 112. The data collected by the detector 120 detecting the secondary beam 112 is related to the line integral of the interactions along the path of the secondary beam 112. In other embodiments, instead of 90°, the detector 120 may be placed at any angle relative to the x-ray source 102. In some embodiments, the system 10 may be reconfigured during an imaging session to a mode to investigate detail agent distribution in a more restricted volume.

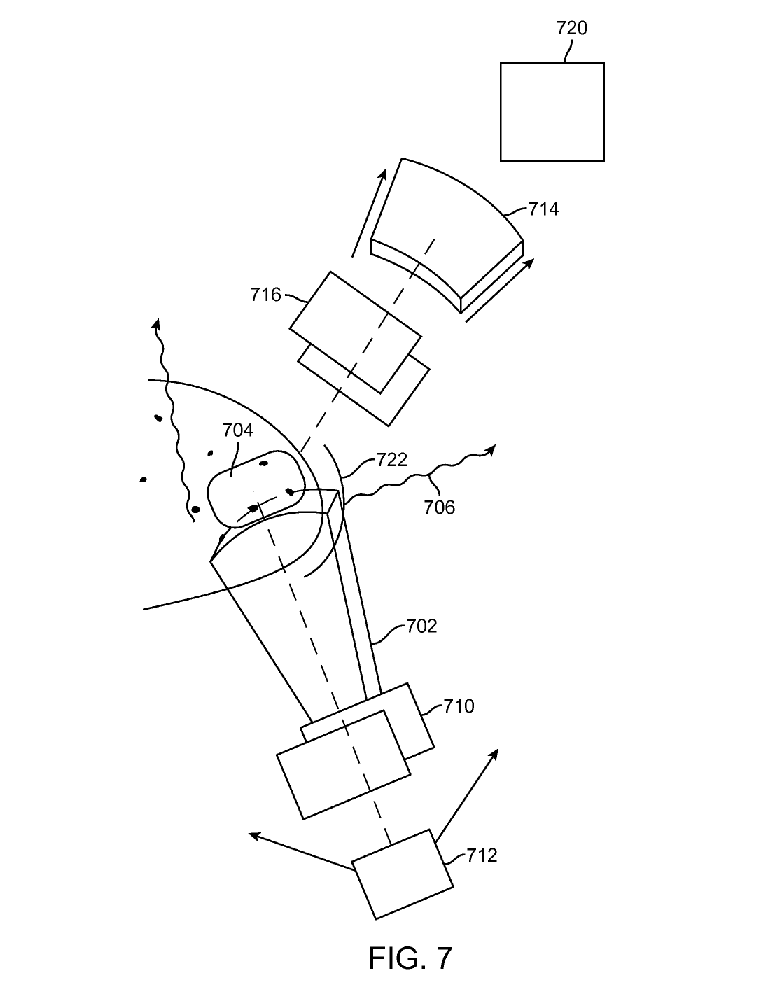

In any of the embodiments described herein, the excitation beam 104 may be a planar beam, and the secondary beam may also be a planar beam (excitation planar beam-secondary planar beam detection mode). FIG. 7 illustrates a detection system that utilizes excitation planar beam and detected secondary planar beam in accordance with some embodiments. In this embodiment, both excitation beams and secondary beams are each collimated by a planar collimator. In particular, the excitation beam 702 is emitted from x-ray source 712 (which may be an example of the source 102 of FIG. 1) and collimated by a first collimator 710 (which may be an example of the collimator 106 of FIG. 1) into excitation planar beam 702. The excitation planar beam 702 may have monochromatic or multi-chromatic spectral characteristics. The excitation planar beams' energy and intensity may also be optimized through Bragg diffraction, crystal selection, and/or collimation. The secondary beams 706 are generated by interaction of the excitation beam 702 with a voxel 704 of tissue and imaging agent within the target volume. The secondary beams 706 are collimated by a planar collimator 716 (which may be an example of the collimator 114 of FIG. 1), and are received by the detector 720 (which may be an example of the detector 120 of FIG. 1). The data collected by the detector 720 detecting the secondary planar beam 714 is a line integral of the scatter along the path of the excitation beam 702. In other embodiments, multiple beam sources 712 and/or multiple detectors 720 may be used. Mounting multiple excitation beam sources 712 and multiple detectors 720 on a gantry, and collecting data from multiple directions or angles relative to the target volume 704 allows for reconstruction of volumetric image of the radioactive imaging agent distribution within the target volume 704. The reconstruction algorithm would be similar to those used in CT, MRI, and PET.

As describe above, in some embodiments, the source beam may be positioned (e.g., translated) during an examination process. In other embodiments, the source beam may be stationary and the detector beam may be translated. In some embodiments, the movement (e.g., translation) of the source 102 and/or the movement (e.g., translation) of the detector, may be used with a planar fan detection (FIG. 6), with a planar fan source, or with both a planar fan detection and a planar fan source (FIG. 7), to thereby collect data over entire volumes, or sub volumes of the target. In any of the embodiments described herein collimation ports for the detector side and/or collimation port for the source side, may be provided in combination with the movement of the source and/or movement of the detector to further enhance the utility of data collection.

In any of the embodiments described herein, the imaging data received from detecting the secondary beams 112 may be reconstructed into a volumetric image using a technique that is similar to CT reconstruction. In a CT reconstruction, the radiation source configured to deliver a fan beam (or any other beam shape) is rotated to different gantry angles, and image data of a target region are obtained at different gantry angles. The image data are used to determine an integral of data at any given point in the target volume, and a volumetric image is constructed. In some embodiments (involving secondary beam at an angle relative to the incidental beam), to construct a volumetric image, similar technique is used (i.e., obtaining image data from different positions relative to the target region, e.g., at different angles, and using reconstruction algorithm to reconstruct an image). However, the difference is that in a standard CT reconstruction algorithm, it is designed for reading the image data of photonic transmission through the target volume, versus that in the current embodiments, the reconstruction algorithm is configured to correct for difference in photonic energy of the incidental beam at different points along the beam path, as well as degradation/changes of the secondary radiation as it travels through the target volume (post scattering).

In the above embodiments, the excitation beam 104 and the secondary beam 112 have been described as having a “pencil” or planar configuration. In other embodiments, the excitation beam 104 and/or the secondary beam 112 may have different configurations. For example, in other embodiments, one or both of the beams 104, 112 may have a customized cross-sectional shape and size, in which cases; the beam does not have a planar configuration or a pencil-like configuration. In some cases, the shape of the beam may be defined by three dimensional configurations that are extensions of pencil and planar beam shapes. Differential dimensional configurations of excitation and secondary beams 104, 112, and their subsets, can be implemented in varieties of different combinations for medical imaging under different circumstances.

There are many possible configurations to the system 10 and methods described herein. One possible configuration is the choice of the imaging agent, which may be naturally occurring or injected. The considerations in choosing an imaging agent include sufficient contrast, excitation radiation cross section (e.g., the effective diameter of the atom that x-ray is interacting with), and detectable characteristic radiation. Other possible configurations may be achieved by using different configurations for the excitation beam 104, different configurations for the secondary beam 112, and/or different configurations for the detector collimation 106 and/or 114. In some embodiments, the excitation beam 104, the secondary beam 112, and/or the collimator(s) 106, 114 may be configured reduce or minimize background photons from the source 102, so that photons from mainly a defined voxel (with certain location, size, and/or shape), and not the source 102, is used. Other possible configurations may be achieved by using different energy spectrums of the excitation beam 104 and/or different intensities of the excitation beam 104. By choosing a proper energy spectrum of the excitation beam 104 and the proper intensity of the excitation beam 104, the system may provide better data and unique recognition of the imaging agent. For example, a mono-energetic excitation beam with energy characteristics different from the characteristics of the secondary radiation from imaging agent may be desirable in some applications. The radiation source 102 may be generated from a radioactive source, Bragg diffraction from an x-ray tube, or any other type of radiation generator that produces a well collimated beam with a controlled energy spectrum (e.g., mono-energetic beam with one or more energies). Other possible configurations may be achieved by using different detectors with different energy resolutions, or detectors with different detector efficiencies. For example, detector efficiency may be chosen to provide sufficient sensitive and accurate measurements. Other factors to consider while configuring the system may include the consideration of limiting the radiation dose given to a patient in a clinical setting to be within a tolerable level yet produces acceptable imaging results. Other factors to further consider while configuring the system may include broader spectrum radiation or varying different quantum attributes of the incidental radiation energy.

Also, in other embodiments, the detector 120 may have an arc configuration. For example, in some cases, the arc of the detector 120 may partially circumscribe an object under examination, wherein the arc may extend at least 90° circumferentially, or more preferably, at least 180° circumferentially. In other embodiments, the detector 120 may extend 360° circumferentially. In such cases, the detector 120 has a ring configuration with an opening in the middle for accommodating the object under examination.

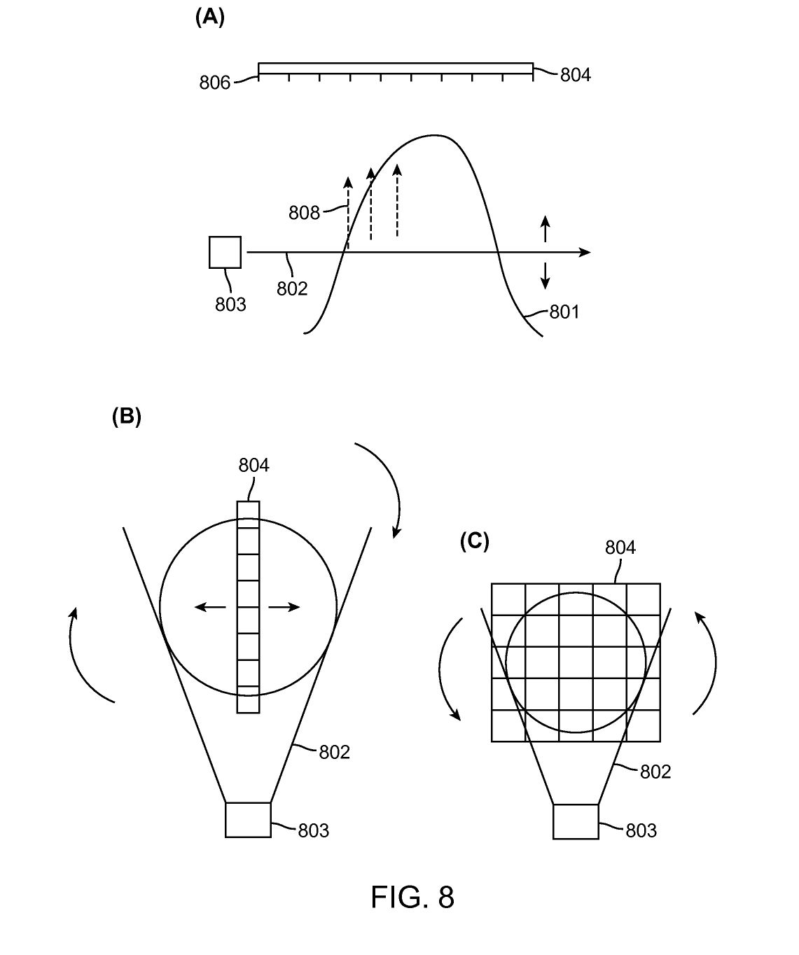

In some embodiments, the system 10 may be configured to produce CT images in high efficiency. As shown in FIG. 8, an exemplary system is configured to produce 3D CT images of a target volume (e.g., a breast). An x-ray source 803 (which may be an example of the source 102 of FIG. 1) is configured to project toward a target volume 801, an incidental x-ray beam 802 that can be collimated in different shapes. In this exemplary configuration, a beam 802 is planar. The x-ray beam 802 is projected across the target volume 801, producing secondary x-ray 808, the path of which is perpendicular to the path of the x-ray beam 802 (e.g., at a 90° angle to the x-ray source 803). The secondary x-ray 808 is collimated by collimator 806 (which may be an example of the collimator 114 of FIG. 1) and detected by detector 804 (which may be an example of the detector 120 of FIG. 1). The detector 804 detects projection data which is the integrated scattering data that is along the path of secondary x-ray 808 through the portion of the target volume 801 that is between the path of the incidental beam 802 and the detector 804. In order to receive more projection data to reconstruct 3D CT image, the x-ray source 803 may move toward the detector 804 (See movement arrow pointing towards the detector 804 in FIG. 8 (A)). The detector 804 can be a small strip detector (See FIG. 8 (B)), or a large area detector (See FIG. 8(C)). In FIG. 8 (B), where a strip detector is used, the detector 804 may be moved left or right as shown in the figure. Also, the x-ray source 803 may rotate partially or completely (e.g., 180°-360°) around the target volume 801. The x-ray source 803 may also \move toward or away from the detector 804, as discussed. Alternatively, the strip detector 804 may be oriented 90° from that shown in the figure, and be moved up and down (i.e., in a direction that is parallel to the axis of the beam 802). In FIG. 8(C), where an area detector 804 is used, the x-ray source 803 may rotate partially or completely (e.g., 180°-360°) around the target volume 801, and/or may move toward or away from the detector 804. The collected projection data can then be used to reconstruct a 3D image of the target volume 801. A modification of the standard CT reconstruction algorithm is employed to reconstruct the 3D image, since the standard CT reconstruction algorithm is designed for reading the image data of photonic transmission through the target volume, versus that in the illustrated embodiments, the reconstruction algorithm must correct for difference in photonic energy of the incidental beam at different points along the beam path, as well as degradation/changes of the secondary radiation has it travels through the target volume (post scattering).

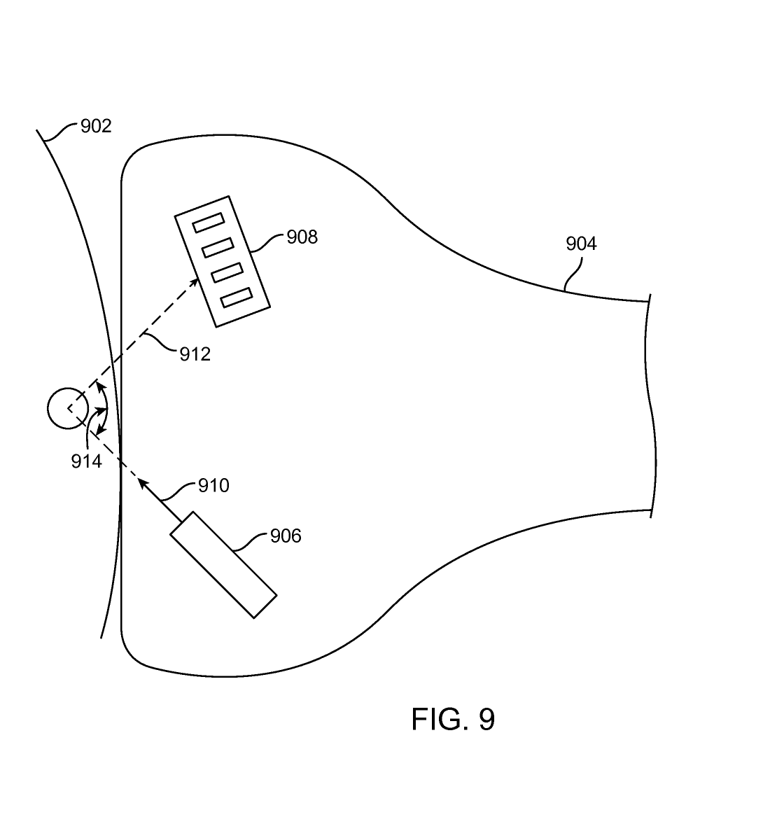

In other embodiments, an apparatus may be provided that includes both the x-ray source and the detector in the same probe to form a compact x-ray imaging device, similar to an ultrasound probe wherein the ultrasound emitter and the detector are included within the same probe housing. As shown in FIG. 9, the radiation source 906 and detector 908 are placed within a probe 904, which is configured for scanning a target volume 902. The radiation source 906 and the detector 908 can be placed at an angle 914 relative to each other. The angle 914 is approximately 90° (e.g., 90°±10°). In other embodiments, the angle 914 may be different from 90°, and may be a value that is anywhere between 0° and 180° and more preferably between 45° and 135°). During use, the excitation radiation beam 910 is emitted towards the target volume 902, and secondary radiation beam 912 is detected by detector 908. Signals from the detector 908 may be used to obtain information about the target volume 902, as similarly discussed herein.

As described in some of the above embodiments, the source collimator may be configured to provide a plurality of ports to create multiple source beams, and/or the detector collimator may be configured to provide a plurality of ports to create multiple secondary (analysis) beams. It should be noted that each port (at the source collimator or the detector collimator) may be configured (e.g., by operating the collimator) to produce a beam having any desired cross sectional shape, which may or may not vary in the longitudinal direction. In some embodiments, the beams (e.g., pencil beams) from the ports (e.g., source collimator ports, or detector collimator ports) may or may not overlap in the longitudinal direction. Also, in some embodiments, the beams (e.g., pencil beams) from the ports may be essentially parallel or they may come to a focus at some arbitrary longitudinal position. In some cases, there may be more than one focal point from different beams. In any of the embodiments described herein, each port may be configured to provide an arbitrary cross sectional cone, focusing at infinity or at certain longitudinal position. In different embodiments, the source supplying radiation to the ports may have different properties, such as different central quantum energy or spectra, different intensity, etc. Also, in different embodiments, the detector for receiving beams from different ports may have different properties, such as sensitivity to intensity, sensitivity to secondary quanta energy, etc.

Also, although several examples of the examination system have been described in different embodiments above, it should be noted that the system for examination subject is not limited to the examples described, and that the system may have different configurations in different embodiments. By means of non-limiting examples, possible configurations for the examination system may include the following in different embodiments:

(A) One or more beam sources and one or more detectors;
(B) A pencil beam source configured to provide a pencil beam, and a detector collimator with a single port configured to provide a secondary beam for detection by the detector;
(C) A pencil beam source configured to provide a pencil beam, and a detector collimator with a multiple ports configured to provide multiple secondary beams (e.g., fan beams, focused cone beams, or unfocused cone beams) for detection by the detector;
(D) A source collimator with a single port configured to provide a beam (e.g., fan beam, focused cone beam, or unfocused cone beam), and a detector collimator configured to provide a pencil secondary beam for detection by the detector;
(E) A source collimator with a multiple ports configured to provide multiple beams (e.g., fan beams, focused cone beams, or unfocused cone beams), and a detector collimator configured to provide a pencil secondary beam for detection by the detector;
(F) A source collimator with a single port configured to provide a beam (e.g., fan beam, focused cone beam, or unfocused cone beam), and a detector collimator with a single port configured to provide a secondary beam (e.g., fan beam, focused cone beam, or unfocused cone beam) for detection by the detector;
(G) A source collimator with a single port configured to provide a beam (e.g., fan beam, focused cone beam, or unfocused cone beam), and a detector collimator with multiple ports configured to provide multiple secondary beams (e.g., fan beams, focused cone beams, or unfocused cone beams) for detection by the detector;
(H) A source collimator with multiple ports configured to provide multiple beams (e.g., fan beams, focused cone beams, or unfocused cone beams), and a detector collimator with a single port configured to provide a single secondary beam (e.g., fan beam, focused cone beam, or unfocused cone beam) for detection by the detector; or
(I) A source collimator with multiple ports configured to provide multiple beams (e.g., fan beams, focused cone beams, or unfocused cone beams), and a detector collimator with multiple ports configured to provide multiple secondary beams (e.g., fan beams, focused cone beams, or unfocused cone beams) for detection by the detector.
In any of the embodiments described above, the source may provide beam(s) that is focused, and the secondary beam(s) may be unfocused. In other embodiments, the source may provide beam(s) that is unfocused, and the secondary beam(s) may be focused. In still other embodiments, the source may provide beam(s) that is focused, and the secondary beam(s) may also be focused. In further embodiments, the source may provide beam(s) that is unfocused, and the secondary beam(s) may also be unfocused. Also, in any of the embodiments described herein, any of the beams (source beam or secondary beam) may be a partial beam (e.g., a partial fan beam) or a full beam (e.g., a full fan beam). A full beam is a beam that is wide enough to cover the entire width of an object of interest, and a partial beam does not.

The various configurations of source and detection described herein may produce information about local points, points along a line, points on a surface, or points in a volume. Such information may be obtained from the data collected by data processing. In some embodiments, this processing may involve CT like reconstruction. In other embodiments, there is no requirement for CT like reconstruction. In either case, algorithms may be provided to process and/or interpret the data to present the information of interest. Also, in any of the embodiments described herein, the data collected using the detector(s) may be processed to produce information of interest such as physical information, and/or functional information. The physical information and/or functional information may be in a one, two, or three-dimensional space domain and/or in a temporal domain. In some embodiments, if the physical information or functional information are generated in a three-dimensional space domain and over time, then such information may be called 4d physical medical characterization or 4d functional medical characterization.

It should be noted that the system and method for examining an object described above are not limited to using x-ray, and that similar techniques may be implemented using other imaging modalities. For example, the object examination techniques described herein may be implemented using a PET system, a CT system (such as a CBCT system), a nuclear imaging system, a magnetic resonance imaging system, a line scan imaging system, etc.

Those skilled in the art will appreciate that various other modifications may be made within the spirit and scope of the claimed invention. All these or other variations and modifications are contemplated by the inventors and within the scope of the claimed invention.

What is claimed:

1. An apparatus to examine a target volume in a patient, comprising:
an x-ray source generating a first x-ray beam targeting the target volume; and
a detector which is placed at an angle less than 180 degrees relative to a beam path of the first x-ray beam to receive a second x-ray beam generated from the first x-ray beam interacting with the target volume.

2. The apparatus of claim 1 further comprising a first collimator between the x-ray source and the target volume

3. The apparatus of claim 2, wherein the first beam is defined by the first collimator.

4. The apparatus of claim 2, further comprising a second collimator between the target volume and the detector.

5. The apparatus of claim 4, wherein the second beam is defined by the second collimator.

6. The apparatus of claim 1, further comprising a source of agent for delivery to the target volume.

7. The apparatus of claim 6, wherein the agent is radioactive.

8. The apparatus of claim 1, wherein the first x-ray beam is quasi mono-energetic.

9. The apparatus of claim 1, wherein the detector comprises a photo spectrum sensitive detector.

10. The apparatus of claim 1, wherein the angle is a value that is anywhere between 30 degrees and 170 degrees.

11. The apparatus of claim 1, wherein the second beam comprises a photo absorption component and a Compton scatter component.

12. The apparatus of claim 1, further comprising a processor for reconstructing a volumetric image of the target volume using examination data derived from signals generated by the detector.

13. A method to image a target volume in a patient, comprising:
directing a first x-ray beam generated from an x-ray source at the target volume, wherein a second x-ray beam is generated by an interaction of the first x-ray beam with the target volume;
detecting the second x-ray beam using a detector that is placed at less than 180 degrees relative to a path of the first x-ray beam; and
obtaining spatial and temporal information of the target volume using the detected second x-ray beam.

14. The method of claim 13, further comprising collimating the first x-ray beam before the first x-ray beam reaches the target volume.

15. The method of claim 14, further comprising collimating the second x-ray beam before the second x-ray beam is detected by the detector.

16. The method of claim 13, wherein the target volume comprises human tissue and an administered agent.

17. The method of claim 16, wherein the agent is radioactive.

18. The method of claim 13, wherein the first x-ray beam is mono-energetic.

19. The method of claim 13, wherein the detector is a photo spectrum sensitive detector.

20. The method of claim 13, wherein the angle is a value that is anywhere between 30 degrees and 170 degrees.

21. The method of claim 13, wherein the second x-ray beam comprises a photo absorption component and a Compton scatter component.

22. The method of claim 13, wherein the temporal information is obtained by continuously detecting the second x-ray beam.

23. The method of claim 13, further comprising generating a volumetric image using the spatial and temporal information.
